(19) United States
(12) Patent Application Publication
(10) Pub. No.: US 2003/0199101 A1
(43) Pub. Date: Oct. 23, 2003

(54) Chlorine test reagent, and reagent storing, packaging and dosing

(76) Inventor: Albert A. Diggens, Onekama, MI (US)

Correspondence Address: Brian M. Dingman, Esq., Mirick, O'Connell, DeMallie & Lougee, LLP, 1700 West Park Drive, Westborough, MA, 01581-3941, US

(21) Appl. No.: 10/418,555

(22) Filed: Apr. 18, 2003

Related U.S. Application Data
(60) Provisional application No. 60/373,675, filed on Apr. 18, 2002.

(51) Int. Cl. G01N 33/00

(52) U.S. Cl. 436/166; 436/124

(57) ABSTRACT

A shelf-stable, pre-measured reagent for a chlorine determination test. The reagent includes at least a source of iodide and a pH buffer packaged into a tablet. The tablet is packaged in a plastic-lined metal foil envelope that completely surrounds the tablet, to inhibit air infiltration into the package.

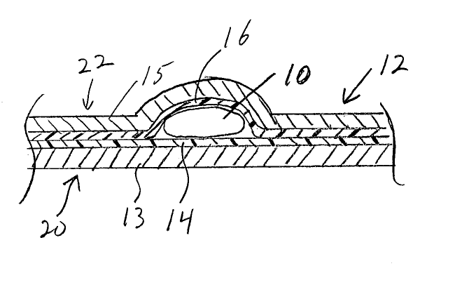

### CROSS REFERENCE TO RELATED APPLICATION

This application claims priority of Provisional application serial No. 60/373,675, filed on Apr. 18, 2002.

### FIELD OF THE INVENTION

This invention relates to a test reagent for determination of chlorine in solution.

### BACKGROUND OF THE INVENTION

Potassium iodide is used as a reagent in an analytical method for the determination of total chlorine. However, potassium iodide is unstable, and in the presence of oxygen in air it forms iodine. It is important to the accuracy of the measurement that the reagent remains in its pure state for long periods of time of months or more. Also, the chlorine determination test requires that a known amount of the reagent be metered into the sample in a fixed and reproducible way.

This reagent is specifically useful for the determination of chlorine, and as most chlorine determinations are accomplished in the field outside of the laboratory environment, the use of liquid reagents is inconvenient. Also the product will be sold through a large number of distributors. A long shelf life of the reagent is thus of great importance to commercial success.

Liquid reagents of the same reagent concentration are stable, even when stored in dark brown glass bottles, for only a few days to weeks, making this type of packaging unsuitable for stocking the product. This is mainly due to the reaction of iodide with oxygen dissolved in the water with which the liquid reagent is made. Also, when liquid reagent is used, the reagent must be metered into the liquid, which is costly and inconvenient, particularly in the field.

### SUMMARY OF THE INVENTION

The measuring device in the chlorine determination test consists of a two electrodes. The voltage (potential) between these two electrodes is measured on a high impedance voltmeter. One electrode is manufactured from platinum, and the other from an iodide ion responsive composite material. The active ingredients of the dry reagent consist of potassium iodide and an appropriate pH buffer to pH 4.

The mechanism of the measurement is as follows:

1. The reagent is added to the sample containing chlorine, and the following reaction occurs:

2I⁻+Cl₂. . . goes to . . . I₂+2Cl⁻

It is necessary for chemical completion that this reaction occurs at around pH 4.0. The amount of chlorine in the sample is equivalent to the amount of iodine produced in this reaction. Thus, an accurate measurement of iodine in the reacted solution will result in a determination of the amount of chlorine in the sample.

2. The platinum electrode measures the redox potential of the reaction above in the following manner

Eₚₗₐₜᵢₙᵤₘ=Eₒ−RT/2F log(iodine/iodide²)

3. The iodide electrode measures the activity of the iodide in the sample as follows

E_{iodide}=Eₒ−RT/2F log iodide ion

4. The difference in potential between these two electrodes is thus:

E_{(platinum minus iodide)}=Eₒ−RT/2F log iodine.

Thus the electrode output signal is directly proportional to the concentration of iodine, and thus the concentration of the original chlorine.

### BRIEF DESCRIPTION OF THE DRAWING

Other objects, features and advantages will occur to those skilled in the art from the following description of the preferred embodiment, and the accompanying figure, which shows an inventive reagent tablet within the inventive packaging.

### DETAILED DESCRIPTION OF THE PREFERRED EMBODIMENT

The invention comprises a means of having a long shelf life of potassium iodide without the formation iodine. The invention further comprises a means of discrete addition of a fixed amount of iodide and pH buffer components to a test solution.

The invention comprises packaging the iodide, appropriate pH buffers to achieve a pH of about 4, and tableting aids such as binding and dissolution excipients, into tablet form, and packaging the tablets into an air-tight foil container. The figure shows tablet 10 of the invention packaged in plastic-lined, metal-foil packaging 12. Packaging 12 comprises heat-sealed sheets of plastic-lined aluminum foil, formed around the tablets. For example, bottom sheet 20 comprises aluminum foil layer 13 lined with plastic sheet 14, and top sheet 22 comprises aluminum foil layer 15 lined with plastic sheet 16. In the preferred embodiment, the aluminum foil sheets are about 1-1.5 mils thick, and are lined with a modified vinyl plastic coating. The plastic coating acts to seal pinholes in the aluminum foil, and heat seals the two sheets together through the use of heated rolls. In the packaging process the tablets are first pressed with a rotary tablet machine and then packaged by a strip packaging machine in which the tablets are inserted between the two foil webs which are then heat sealed together to form an envelop such as shown in the drawing. Each package can include one or more tablets packaged in this manner. Alternatively, the packaging can contain the pre-measured dose of ingredients in powdered form.

The chemical reaction described above clearly illustrates the need for a reagent that contains no iodine, as this would be measured as a background “chlorine” and thereby give rise to erroneous results. To ensure adequate analytical accuracy it is also necessary to add this reagent in fixed and consistent amounts. The invention satisfies both these requirements. The test comprises measuring a known amount of liquid containing chlorine, adding an inventive tablet, and shaking or stirring to dissolve the tablet. The electrodes are then placed into the solution, and the potential difference measured in a known fashion.

The iodide in the reagent will be oxidized by the oxygen in air, which would convert some or all of the iodide present in the reagent to iodine, thus causing problems with accuracy and detection limits. Tabletizing the reagent, and packaging the tablets in air-tight foil, minimizes the possibility that oxygen, from the atmosphere, will react with the iodide in the reagent. Also, the single dose reagent aspect of this approach makes the measurement more convenient for the customer and less prone to reagent measurement error.

Using this method of packaging satisfies the conditions of preventing air oxidation of the tablet or powder, and provides a precise and consistent means of metering the reagent and the pH buffer into the sample. Also, the aluminum of the preferred aluminum foil for the packaging helps to maintain the iodide in the necessary reduced form.

The preferred tablet size is about 110-120 mg for a 20 ml sample. Each tablet contains a specific amount of the following chemicals, although the invention is not specifically restricted to these chemicals.

Tablet/ Reagent formulation (the relative amounts of each ingredient are selected as necessary, as is known in the art).

1. Potassium iodide

2. pH buffering agents to pH about 4

3. Binding and dissolution excipients (tableting aids known in the art)

Tests have shown that this packaging keeps the tablet stable for at least nine months, and it is anticipated that the packaged tablets will last for many years in this stable form.

Other embodiments will occur to those skilled in the art and are within the following claims:

What is claimed is:

1. A shelf-stable, pre-measured reagent for a chlorine determination test, comprising:
at least a source of iodide and a pH buffer packaged into a tablet;
the tablet packaged in a metal foil envelope that completely surrounds the tablet, to inhibit air infiltration into the package.

2. The shelf-stable, pre-measured reagent of claim 1 wherein the metal foil is lined with plastic, to seal pinholes in the foil.

3. The shelf-stable, pre-measured reagent of claim 2 wherein the packaging comprises two bonded sheets of plastic lined metal foil, the plastic linings of the two sheets being bonded together.

4. The shelf-stable, pre-measured reagent of claim 1 wherein the source of iodide is potassium iodide.

5. The shelf-stable, pre-measured reagent of claim 1 wherein the pH buffer accomplishes a test pH of about 4.

6. The shelf-stable, pre-measured reagent of claim 1 wherein the metal foil comprises aluminum.

7. The shelf-stable, pre-measured reagent of claim 1 wherein the tablet has a weight of about 110-120 mg.

8. A shelf-stable, pre-measured reagent for a chlorine determination test, comprising:
at least potassium iodide and a pH buffer that accomplishes a test pH of about 4, packaged into a tablet;
the tablet packaged in a plastic-lined aluminum foil envelope that completely surrounds the tablet, to inhibit air infiltration into the package.
